(19) United States
(12) Patent Application Publication
(10) Pub. No.: US 2014/0336626 A1
(43) Pub. Date: Nov. 13, 2014

(54) MEDICAL ASSEMBLY USING SHORT PULSE FIBER LASER

(71) Applicant: ADVALUE PHOTONICS, INC., Tucson, AZ (US)

(72) Inventors: Shibin Jiang, Tucson, AZ (US); Jihong Geng, Tucson, AZ (US); Qing Wang, Tucson, AZ (US)

(21) Appl. No.: 13/891,487

(22) Filed: May 10, 2013

(51) Int. Cl. A61B 18/26

(52) U.S. Cl. CPC A61B 18/26
USPC 606/2.5

(57) ABSTRACT

A method of ablating a solid substance within a mammalian body is presented. The method includes generating a superheated zone within the body using a fiber lasing assembly that emits pulses at a pulse repetition rate of 1 kHz to 500 kHz, where each pulse has a wavelength from 1.7 micron to 2.2 micron, a pulse width from 2 ns to 800 ns, and a pulse energy from 0.05 mJ to 2 mJ. The fiber lasing assembly used further includes a seed laser and an amplifier optically connected to the seed laser by an Ho-doped, Tm-doped, or Ho Tm co-doped fiber.

### TECHNICAL FIELD

The present invention relates generally to medical procedures using short pulse fiber lasers, and more particularly to those procedures using fiber lasers with a pulse width shorter than 1 micro second.

### BACKGROUND

Lasers are used in a wide variety of medical applications, such as cancer diagnosis and treatment, hair removal and tattoo removal, dermatology, lithotripsy, ophthalmology, prostatectomy, and surgery. While the preferred type of laser for each medical application depends greatly on the specific requirements of the particular procedure, generally speaking, the major requirements for any medical application includes effectiveness, ease of use, high reliability, good repeatability, and safety. Medical professionals are always looking for lasers that better meet these requirements and, in response, the laser industry is constantly improving laser technology.

For example, laser lithotripsy started the development in 1979 to remove kidney stones. Specifically, in laser lithotripsy a laser beam comes in direct contact with kidney stones, causing the stones to fragment. This technology can provide a minimally-invasive treatment, requiring general anesthesia and allowing the patient to go home the same day. Several US patents have been directed to lasers that can be used in lithotripsy. For example, U.S. Pat. No. 5,059,200 to Tulip disclosed a Nd:YAG laser operating at 1.44 micron wavelength, which emitted 27 pulses per second and which was delivered to the body through a quartz fiber cable passing through the interior of an endoscope. Additionally, U.S. Pat. No. 5,071,422 to Watson, et al. disclosed pulsed dye laser at visible wavelength to break down calculi, stones, calcified tissue and other materials. Further, U.S. Pat. No. 5,009,658 to Damgaard-Iversen, et al. disclosed method and apparatus for lithotripsy using two spatially and temporally overlapping pulsed laser beams in the 300-450 nm and 600-900 nm wavelength range. Subsequent to these developments, a Ho:YAG laser was identified as an efficient and versatile tool for lithotripsy and thereafter, U.S. Pat. No. 5,860,972 to Hoang disclosed method and devices for detecting, destroying and removing urinary calculi and other similar structures within an animal body using a Ho:YAG laser at near 2.1 micron wavelength.

When lasers are used for lithotripsy, the doctor uses an endoscope (a tube introduced into the body, via the urinary tract) in order to get close to the stone. A small fiber is snaked up the endoscope so that the tip that emits the laser energy can come in contact with the stone. The intense laser energy breaks the stone into increasingly smaller pieces, which can be extracted or flushed out.

More specifically, in laser lithotripsy, laser energy is delivered to the stone via optical fibers and is converted into mechanical energy in the form of a cavitation bubble associated with the occurrence of shock-waves. When the laser energy is absorbed by the absorbing liquid, a rarefaction zone inside the heated volume is formed, which produces compressive and tensile stresses in the liquid. When a large amount of energy is deposited at high laser fluencies, the liquid is heated substantially above the equilibrium vaporization temperature. When the temperature is comparable with the thermodynamic critical temperature, vaporization takes place to form a bubble.

Other ways of generating cavitation voids involve the local deposition of energy, such as an intense focused laser pulse (optic cavitation) or with an electrical discharge through a spark. In such methods, vapor gases evaporate into the cavity from the surrounding medium. As a result, while the cavity is not a perfect vacuum, it has a relatively low gas pressure.

When the bubble is large enough, the cavitation bubble in a liquid collapses due to the higher pressure of the surrounding medium, which releases a significant amount of energy in the form of an acoustic shock wave. It is generally believed that the shock wave plays an important role to break the stones. This process is illustrated schematically in FIG. 1.

In the prior art, the most-widely-used laser source for laser lithotripsy is a pulsed Holmium-doped laser with a pulse width in the hundreds of microseconds. Because of the high absorption of water and urinary calculi, the laser light is efficiently converted from optical energy into thermal energy, and, due to the time scale of the laser pulse width, the dominant mechanism is photothermal interaction along with minor effects of acoustic emission. Further, in most prior art laser lithotripsy applications, a pulse energy of 0.2 and 5 Joules is used. For example, U.S. Pat. No. 5,860,972 to Hoang discloses a Ho:YAG laser with a typical pulse energy of 0.2 and 2.8 Joules, a frequency of 5 and 20 Hz, and a typical pulse duration of 350 microsecond. A relatively long pulse width is needed in order to produce the strong shock wave. In fact, U.S. Pat. No. 5,496,306 to Engelhardt, et al. disclosed a method of laser lithotripsy that utilized pulse stretched Q-stitched solid state lasers. The laser pulse is stretched from hundreds of nanoseconds to 2 microseconds to achieve effective breakup of calculi located within the body. The pulse energy was from 15 mJ to 200 mJ.

Typically in prior art laser lithotripsy applications, the minimum energy is larger than tens of millijoules in order to create the necessary shock wave. For example, U.S. Pat. No. 5,071,422 to Watson, et al. disclosed pulsed dye laser with 25 to 150 mJ pulse energy and 0.5 to 10 microseconds pulse duration. Additionally, U.S. Pat. No. 5,009,658 to Damgaard-Iversen used minimum pulse energy of 17 mJ from two laser wavelengths. In fact the pulse energy of the laser is so high that U.S. Pat. No. 5,860,972 to Hoang disclosed a method to monitor the sparkle and emission of visible light during laser lithotripsy process.

Clearly therefore, safety is an extremely serious concern for the prior art laser lithotripsy. First, there is the risk that the nearby tissue is damaged during the process. But secondly, the high pulse energy of the laser process produces bubbles (gases). Not only are these bubbles a waste of the laser energy, but they can cause serious damage to the body.

In order to provide a safer laser lithotripsy process, U.S. Pat. No. 8,409,176 to Cecchetti discloses a system/method for destruction/ablation of stones, calculi or other hard substances using continuous wave (CW) diode lasers. Commonly commercially available diode lasers at wavelengths of 980 nm, 1470 nm and 1940 nm can be used. But a CW laser is not effective for laser lithotripsy when the laser has the same average power.

As an alternative to laser lithotripsy, shock wave technologies have also been developed over the past several decades, including an electrohydraulic lithotripter, an electromagnetic lithotripter, and a piezoelectric lithotripter. These extracorporeal shock wave lithotripter technologies use a high-powered acoustic wave that is focused onto the kidney stones inside a body. The focused acoustic waves have such high energy that shock wave generation can occur near the kidney stones, thereby resulting in stone fragmentation.

Although shock wave lithotripsy has the important advantage of being performed extracorporeally, exposure to a shock wave dosage sufficient to comminute kidney stones can case several serious adverse effects, such as hematuria and damage to the kidney tissue or nearby structures in the stomach area resulting in renal and perirenal hemorrhage. Thus, shock wave lithotripsy is also associated with substantial risk for the patent.

Clearly therefore, what is needed is a device which can safely and effectively break up kidney stones with minimal risk of complications for the patient.

### SUMMARY OF THE INVENTION

In one implementation a fiber lasing assembly for ablating a solid substance within a mammalian body by emitting pulses is presented. The fiber lasing assembly includes a seed laser and an amplifier optically connected to the seed laser by an Ho-doped, Tm-doped, or Ho Tm co-doped fiber. The fiber lasing assembly can emit the pulses at a pulse repetition rate of 1 kHz to 500 kHz, where each pulse has a wavelength from 1.7 micron to 2.2 micron, a pulse width from 2 ns to 800 ns, and a pulse energy from 0.05 mJ to 2 mJ.

In another implementation, a method of ablating a solid substance within a mammalian body is presented. The method includes generating a superheated zone within the body using a fiber lasing assembly that emits pulses at a pulse repetition rate of 1 kHz to 500 kHz, where each pulse has a wavelength from 1.7 micron to 2.2 micron, a pulse width from 2 ns to 800 ns, and a pulse energy from 0.05 mJ to 2 mJ. The fiber lasing assembly used further includes a seed laser and an amplifier optically connected to the seed laser by an Ho-doped, Tm-doped, or Ho Tm co-doped fiber.

In another implementation, an article of manufacture is presented that has a processor and a computer readable medium having computer readable program code disposed therein for ablating a solid substance within a mammalian body. The computer readable program code includes a series of computer readable program steps to effect generating a first superheated zone within the mammalian body using a fiber lasing assembly that emits pulses at a pulse repetition rate of 1 kHz to 500 kHz, where each pulse has a wavelength from 1.7 micron to 2.2 micron, a pulse width from 2 ns to 800 ns, and a pulse energy from 0.05 mJ to 2 mJ.

In yet another implementation a computer program product encoded in a computer readable medium is presented where the computer program product is useable with a programmable computer processor for ablating a solid substance within a mammalian body. The computer program product includes a computer readable program code that causes the programmable processor to generate a first superheated zone within the mammalian body using a fiber lasing assembly that emits pulses at a pulse repetition rate of 1 kHz to 500 kHz, where each pulse has a wavelength from 1.7 micron to 2.2 micron, a pulse width from 2 ns to 800 ns, and a pulse energy from 0.05 mJ to 2 mJ.

### BRIEF DESCRIPTION OF THE DRAWINGS

Implementations of the invention will become more apparent from the detailed description set forth below when taken in conjunction with the drawings, in which like elements bear like reference numerals.

FIG. 1 is a schematic illustrating the shock wave produced by a high energy laser pulse such as generated by the lasers of the prior art;

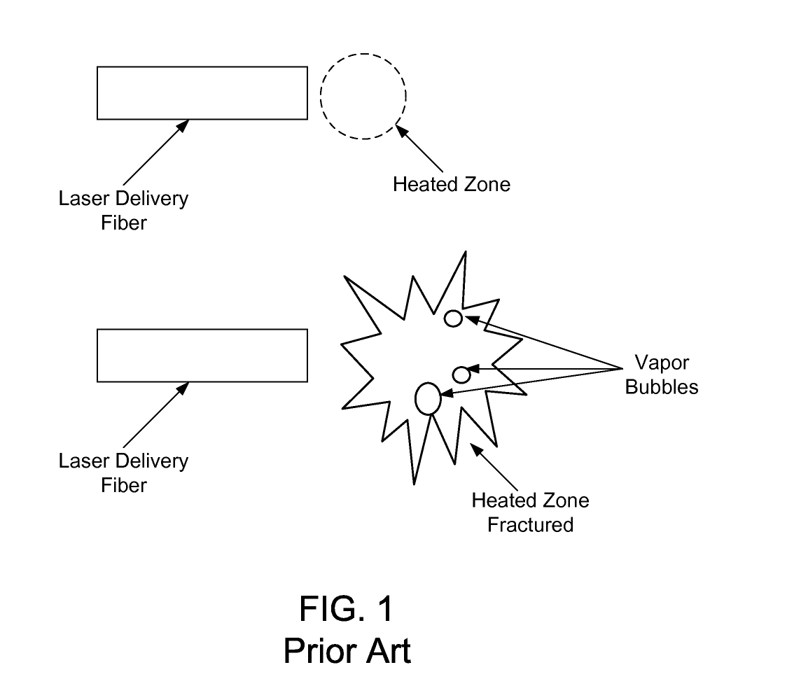

FIGS. 2A-2D are schematics illustrating the process of using a series of superheated zones to fragment a hard substance within a mammalian body; and

FIGS. 3A and 3B are schematics of a 2 micron pulse fiber lasing assembly according to the present invention.

### DETAILED DESCRIPTION OF SPECIFIC EMBODIMENTS

Throughout the following description, this invention is described in reference to specific embodiments and related figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the terms “in one embodiment, “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention that are being discussed.

The present invention includes the use of a short pulse near 2 micron laser for destruction/ablation of stones, calculi or other hard substances within a body. The 2 micron fiber laser has the following characteristics:
1. Laser wavelength from 1.7 micron to 2.2 micron;
2. Pulse width from 5 ns to 800 ns;
3. Pulse energy from 0.05 mJ to 2 mJ; and
4. Pulse repetition rate (also called the pulse repetition frequency) from 1 kHz to 500 kHz.

A 2 micron wavelength is used because water has a strong absorption rate at this wavelength. Specifically, the absorption depth of water near 2 microns is approximately 0.2 mm. Thus, as the human body has a high water content in most areas, the use of a 2 micron laser ensures that the effect of the laser is limited to a small area.

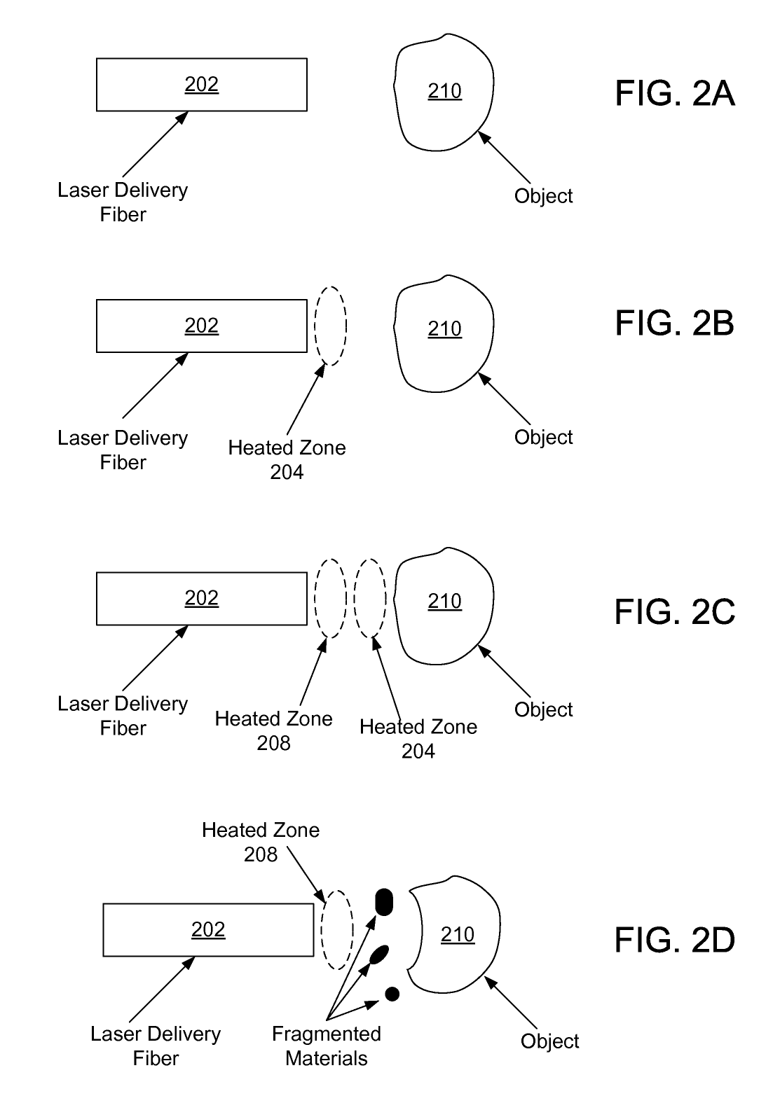

The short pulse near 2 micron laser of the present invention has several advantages over holmium crystal solid state lasers, such as the prior art Ho:YAG lasers, when used for lithotripsy. More specifically, the pulse width of the near 2 micron laser is 2 ns to 800 ns and the pulse energy is only 0.05 mJ to 2 mJ. The Ho:YAG laser, such as disclosed in U.S. Pat. No. 5,860,972 to Hoang, by comparison, has a pulse width of approximately 100 μs and a pulse energy of 200 mJ to 2,800 mJ. Because of this shorter pulse, the water in the human body can absorb the laser energy from the laser of the present invention in a very short period of time—much faster than with a Ho:YAG laser. This small, heated zone can be called a “superheated zone,” comprising such a small volume that is absorbing laser energy over such a short time frame that it can't expand during the laser pulse heating and will rapidly enter the compression stage. Moreover, because of the low pulse energy, the superheated zone will fragment the object without causing vapor. Since the laser delivery fiber is almost touching the object (stones, calculi or other hard substances), the superheated zone will push the object, which aids in fragmentation. Moreover, as the laser of the present invention has a repetition rate of 1 kHz to 500 kHz instead of the typical repetition rate of 5 Hz to 20 Hz for Ho:YAG solid state lasers, a first superheated zone will be pushed toward the object by a following newly created superheated zone before the first has time to propagate away. This process is illustrated schematically in FIGS. 2A-2D. As can be seen, delivery fiber 202 is almost in contact with object 210. As superheated zone 204 absorbs laser energy it is pushed into object 210 by the next superheated zone 208, thereby causing object 210 to fragment into pieces 206.

Further advantages of a short pulse near 2 micron laser as disclosed herein includes that it does not produce vapor bubbles. As a result the lithotripsy procedure is much safer for patients and much more energy efficient than procedures performed with the prior art, commonly used Ho solid state laser. Safety is further increased by the use of a lower pulse energy, which also has the additional advantage of fragmenting the object into smaller pieces, facilitating removal of the same. Fragmentation is further aided by the fact that both the direct heating on the object from the laser and the push force are acting at the same time. Additionally, fiber lasers, such as that of the present invention, are much more reliable and compact than free-space solid state lasers used in the prior art. The fibers of the fiber lasers are fusion spliced together so that there are no misalignment issues as typically associated with free-space lasers. As will be appreciated, reliability is an extremely important feature for medical applications.

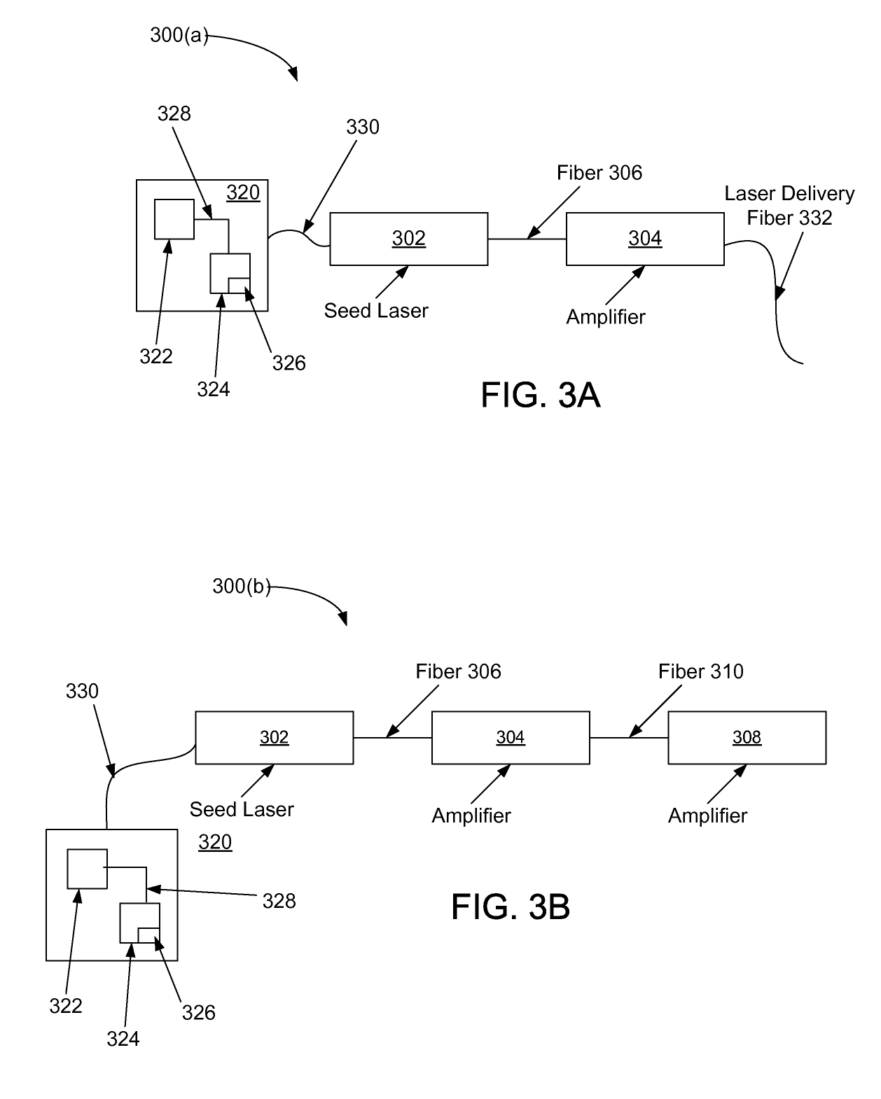

Turning now to FIGS. 3A and 3B, schematics of two embodiments of a short pulse near 2 micron lasing device as encompassed by the present invention is presented. As can be seen in the illustrated embodiment of FIG. 3A, laser system 300(a) comprises a seed laser 302, an amplifier 304 optically connected by fiber 306, where fiber 306 is fusion spliced with seed laser 302 and amplifier 304, and an optical delivery fiber 332. In certain embodiments seed laser 302 is a pulsed fiber laser while in other embodiments it is a modulated fiber pigtailed semiconductor laser. Fiber 306 is a Tm-doped fiber, Ho-doped fiber, or a Tm—Ho co-doped fiber as described in U.S. Pat. No. 8,265,107 to Jiang, et al. or U.S. Patent Publication No. 20120269210 to Jiang, et al., both of which are incorporated herein by reference under 37 C.F.R. §1.57 in their entireties. In certain embodiments, fiber 306 is silicate or germanosilicate glass. In other embodiments, fiber 306 is silica, germante, fluoride, or tellurite glass.

In certain embodiments, laser system 300(a) further comprises computing device 320 interconnected with seed laser 302 via a data communication link 330, wherein computing device 320 comprises a computer processor 322 in communication via data communication link 328 with non-transitory memory 324 having computer program instructions 326 encoded thereon. In such embodiments, computing device 320 is selected from the group consisting of a work station, personal computer, smart phone, or other like device from which information can be stored and/or processed. In such embodiments, computer readable medium 324 comprises a magnetic information storage medium, an optical information storage medium, an electronic information storage medium, and the like. By “magnetic storage medium,” it is meant, for example, a device such as a hard disk drive, floppy disk drive, or magnetic tape. By “optical information storage medium,” it is meant, for example, a Digital Versatile Disk (“DVD”), High-Definition DVD (“HD-DVD”), Blu-Ray Disk (“BD”), Magneto-Optical (“MO”) disk, Phase-Change (“PC”) disk, etc. By “electronic storage media” it is meant, for example, a device such as PROM, EPROM, EEPROM, Flash PROM, compactflash, smartmedia, and the like. In certain embodiments, memory 324 comprises a magnetic information storage medium, and optical information storage medium, an electronic information storage medium, and the like.

As can be seen in the embodiment of FIG. 3B, laser system 300(b) includes a second amplifier 308 that is optically connected to amplifier 304 by a second fiber 310. In certain embodiments amplifier 308 is the same as amplifier 304 and in other embodiments it is different from amplifier 304. As with fiber 306, fiber 310 can be a Tm-doped fiber, Ho-doped fiber, or a Tm—Ho co-doped fiber. In certain embodiments fiber 310 is the same as fiber 306 and in other embodiments it is different from fiber 306. As will be appreciated, while the embodiment of FIG. 3B only depicts two amplifiers, this is intended to be illustrative and not limiting. In certain embodiments three or more fiber amplifiers may be used.

While the forgoing discussion of the present invention has been in terms of the use of a short pulse near 2 micron laser for laser lithotripsy, other medical applications for said laser are well within the scope of the present invention. By way of example and not limitation, the short pulse near 2 micron laser disclosed herein can be used to form liquid jets and acoustic streams to break down blood clots. In the prior art, U.S. Pat. No. 6,022,309 to Celliers, et al. disclosed a catheter-based device for generation of an ultrasound excitation in the biological tissue. Pulsed laser light is guided through an optical fiber to provide the energy for producing the acoustic vibrations. The optical energy is deposited in a water based absorbing fluid, e.g. saline, thrombolytic agent, blood, or thrombus, and generates an acoustic impulse in the fluid through thermoelastic and/or thermodynamic mechanism. Further, U.S. Pat. No. 7,815,632 to Hayakawa, et al. disclosed a laser induced liquid jet generating device and U.S. Patent Publication WO201115328 A1 from Yoh, et al. disclosed a microjet drug delivery system using pulsed lasers. In all of these disclosures, optical energy is converted into mechanical energy or acoustic energy and the residual optical laser energy is wasted. The short pulse near 2 micron laser disclosed herein is advantageous in each of these applications because the lower pulse energy results in a safer, more energy efficient procedure and fiber lasers are inherently more reliable than typical solid state lasers. As described, the 2 micron wavelength of the present laser has a strong absorption. Further, a 2 micron fiber laser can emit a laser beam having a diameter of less than 20 microns, meaning that smaller diameter delivery fibers can be used, such as fibers having 50 micron, 80 micron, or 100 micron diameters. A smaller diameter delivery fiber can get into smaller blood vessels.

While the preferred embodiments of the present invention have been illustrated in detail, it should be apparent that modifications and adaptations to those embodiments may occur to one skilled in the art without departing from the scope of the present invention as set forth in the following claims.

What is claimed is:

1. A fiber lasing assembly for ablating a solid substance within a mammalian body by emitting optical pulses, the fiber lasing assembly comprising:
a seed laser; and
a first amplifier optically connected to the seed laser by a first fiber, wherein the first fiber comprises first laser glass doped with a first rare earth oxide selected from the group consisting of holmium oxide and thulium oxide;
wherein the fiber lasing assembly is configured to emit pulses at a pulse repetition rate of 1 kHz to 500 kHz, wherein each pulse has:
a wavelength from 1.7 micron to 2.2 micron;
a pulse width from 2 ns to 800 ns; and
a pulse energy from 0.05 mJ to 2 mJ.

2. The fiber lasing assembly of claim 1, wherein the first rare earth oxide is a combination of holmium oxide and thulium oxide.

3. The fiber lasing assembly of claim 1 further comprising a second amplifier optically connected to the first amplifier by a second fiber, wherein the second fiber comprises a second laser glass doped with a second rare earth oxide selected from the group consisting of holmium oxide and thulium oxide.

4. The fiber lasing assembly of claim 2, wherein the first rare earth oxide is different from the second rare earth oxide.

5. The fiber lasing assembly of claim 2, wherein the first rare earth oxide is the same as the second rare earth oxide.

6. The fiber lasing assembly of claim 1, wherein the seed laser is a pulsed fiber laser.

7. The fiber lasing assembly of claim 1, wherein the seed laser is a modulated fiber pigtailed semiconductor laser.

8. The fiber lasing assembly of claim 1, further comprising a laser delivery fiber.

9. The fiber lasing assembly of claim 1, further comprising:
a computing device in communication with the seed laser, wherein the computing device comprises:
a processor; and
a computer readable medium in communication with the computing device, wherein the computer readable medium has computer readable program code encoded thereon that when executed by the processor cause the fiber lasing assembly to emit the pulses.

10. A method of ablating a solid substance within a mammalian body comprising:
generating a first superheated zone within the mammalian body using a fiber lasing assembly that emits pulses at a pulse repetition rate of 1 kHz to 500 kHz, wherein each pulse has:
a wavelength from 1.7 micron to 2.2 micron;
a pulse width from 2 ns to 800 ns; and
a pulse energy from 0.05 mJ to 2 mJ.

11. The method of claim 10, further comprising generating a second superheated zone within the mammalian body using the fiber lasing assembly, wherein the second superheated zone pushes the first superheated zone towards the solid substance.

12. The method of claim 10, further comprising fragmenting the solid substance without causing vapor.

13. The method of claim 10, further comprising limiting absorption of the pulse energy by body tissue surrounding the solid substance.

14. The method of claim 10, further comprising placing a laser delivery fiber in near contact with the solid substance.

15. The method of claim 10, further comprising providing the fiber lasing assembly comprising:
a seed laser; and
a first amplifier optically connected to the seed laser by a first fiber, wherein the first fiber comprises first laser glass doped with a first rare earth oxide selected from the group consisting of holmium oxide and thulium oxide.

16. An article of manufacture comprising a processor and a computer readable medium comprising computer readable program code disposed therein for ablating a solid substance within a mammalian body, the computer readable program code comprising a series of computer readable program steps to effect:
generating a first superheated zone within the mammalian body using a fiber lasing assembly that emits pulses at a pulse repetition rate of 1 kHz to 500 kHz, wherein each pulse has:
a wavelength from 1.7 micron to 2.2 micron;
a pulse width from 2 ns to 800 ns; and
a pulse energy from 0.05 mJ to 2 mJ.

17. The article of manufacture of claim 16, further comprising a series of computer readable steps to effect generating a second superheated zone within the mammalian body using the fiber lasing assembly, wherein the second superheated zone pushes the first superheated zone towards the solid substance.

18. The article of manufacture of claim 16, further comprising a series of computer readable steps to effect fragmenting the solid substance without causing vapor.

19. The article of manufacture of claim 16, further comprising a series of computer readable steps to effect limiting absorption of the pulse energy by body tissue surrounding the solid substance.

20. A computer program product encoded in a computer readable medium, the computer program product being useable with a programmable computer processor for ablating a solid substance within a mammalian body, the computer program product comprising:
computer readable program code which causes the programmable processor to generate a first superheated zone within the mammalian body using a fiber lasing assembly that emits pulses at a pulse repetition rate of 1 kHz to 500 kHz, wherein each pulse has:
a wavelength from 1.7 micron to 2.2 micron;
a pulse width from 2 ns to 800 ns; and
a pulse energy from 0.05 mJ to 2 mJ.

21. The computer program product of claim 20, further comprising computer readable program code which causes the programmable processor to generate a second superheated zone within the mammalian body using the fiber lasing assembly, wherein the second superheated zone pushes the first superheated zone towards the solid substance.

22. The computer program product of claim 20, further comprising computer readable program code which causes the programmable processor to fragment the solid substance without causing vapor.

23. The computer program product of claim 20, further comprising computer readable program code which causes the programmable processor to limit absorption of the pulse energy by body tissue surrounding the solid substance.
